(19) United States
(12) Patent Application Publication
(10) Pub. No.: US 2020/0261251 A1
(43) Pub. Date: Aug. 20, 2020

(54) GASTRIC POSITIONING DEVICE

(71) Applicant: Covidien LP, Mansfield, MA (US)

(72) Inventors: Olesea Diaz-Chiosa, Minneapolis, MN (US); Stanislaw Marczyk, Minneapolis, MN (US)

(21) Appl. No.: 16/749,915

(22) Filed: Jan. 22, 2020

Related U.S. Application Data
(60) Provisional application No. 62/807,346, filed on Feb. 19, 2019.

(51) Int. Cl. A61F 5/00

(52) U.S. Cl. CPC A61F 5/0089; A61F 2005/002; A61F 5/0079; A61F 5/0036

(57) ABSTRACT

A gastric positioning device includes a distal tip disposed at a distal end portion of an elongate shaft. The distal tip has an asymmetrical geometry including a closed distal end having a blunt apex aligned along a tip axis that is laterally offset from a central longitudinal axis of the elongate shaft. The gastric positioning device may additionally or alternatively include a handle extending from a proximal end portion of the elongate shaft and a force monitoring system including a force sensor for detecting force applied to the distal tip and an indicator for signaling when the force exceeds a pre-set force threshold value.

### CROSS-REFERENCE TO RELATED APPLICATION

This application claims the benefit of and priority to U.S. Provisional Patent Application No. 62/807,346 filed Feb. 19, 2019, the entire disclosure of which is incorporated by reference herein.

### BACKGROUND

Various bariatric procedures have been developed to treat obesity, including, for example, gastric bypass, adjustable gastric banding, and sleeve gastrectomy. The goal in each of these procedures is to reduce stomach capacity to restrict the amount of food that a patient can eat. The reduced stomach capacity, in turn, results in a feeling of fullness for the patient after ingesting a relatively smaller amount of food. Thus, the patient can achieve weight loss.

Sleeve gastrectomy involves transecting a stomach, e.g., using a stapling device or other suitable device, to reduce stomach volume. Sleeve gastrectomy procedures are often aided by the use of a gastric tube, which serves as a guide or template for transecting the stomach to the appropriate configuration while inhibiting inadvertent transection of stomach or esophageal tissue. Once the stomach has been appropriately transected, the gastric tube is removed and a leak test is performed to determine whether there are any areas of extravasation.

In use, the gastric tube may be advanced into a patient's body through an oral cavity and down through the esophagus into the stomach to provide delineation of the antrum of the stomach, irrigation/suction of fluids, and/or a sizing of a gastric pouch. While being advanced, a clinician, aware of the risk of injury, such as perforation, and due at least in part to the circuitous nature of this track, may need to reposition the gastric tube in various orientations until the gastric tube is properly aligned or it bypasses any obstruction(s).

### SUMMARY

In one aspect of the disclosure, a gastric positioning device includes an elongate shaft and a distal tip. The elongate shaft has a proximal end portion and a distal end portion, and defines a central longitudinal axis. The distal tip extends from the distal end portion of the elongate shaft and has an asymmetrical geometry including a closed distal end having a blunt apex aligned along a tip axis laterally offset from the central longitudinal axis.

The tip axis may be parallel to the central longitudinal axis. The distal tip may extend asymmetrically about the tip axis and/or the central longitudinal axis of the elongate shaft. The distal tip may be curved.

In some embodiments, the distal tip includes a beveled edge extending contiguously from the blunt apex. In certain embodiments, the distal tip includes a curved edge.

In some embodiments, the gastric positioning device includes a sail supported on the elongate shaft. The sail is movable relative to the elongate shaft between an unexpanded position in which the sail abuts the elongate shaft and an expanded position in which the sail is bowed outwardly from the elongate shaft.

In some embodiments, the gastric positioning device includes a handle extending proximally from the elongate shaft.

In another aspect of the disclosure, a gastric positioning device includes an elongate shaft, a distal tip, a handle, and a force monitoring system. The elongate shaft has a proximal end portion and a distal end portion, the distal tip extends from the distal end portion of the elongate shaft, and the handle extends from the proximal end portion of the elongate shaft. The force monitoring system includes a force sensor for detecting force applied to the distal tip and an indicator for signaling when the force exceeds a pre-set force threshold value.

In some embodiments, the force sensor is disposed within the handle. In certain embodiments, the force sensor is a strain gauge.

In some embodiments, the force sensor is disposed at the distal tip. In certain embodiments, the force sensor is a piezoresistive sensor. The distal tip may include a receiver disposed therein that is configured to receive electrical signals from the force sensor.

The handle may include a microcontroller disposed therein that is configured to process electrical signals from the force sensor.

In some embodiments, the indicator is a light. The handle may include a power source disposed therein that is operably coupled to the indicator.

In yet another aspect of the disclosure, a method of detecting force at a distal tip of a gastric positioning device by a force monitoring system of the gastric positioning device includes: sensing a force at a distal tip of a gastric positioning device; comparing the force to a pre-set force threshold value; and activating an indicator if the force reaches or exceeds the pre-set force threshold value.

In some embodiments, sensing the force includes transforming mechanical stimuli into an electrical signal, and the method further includes processing the electrical signal into a digital value comparable against the pre-set force threshold value.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure, wherein:

FIG. 1 is a perspective view of a gastric positioning device including a distal tip having an asymmetrical geometry including a beveled edge;

FIG. 2 is an enlarged view of the distal tip of FIG. 1;

FIG. 3 is an enlarged view of a distal tip of the prior art;

FIG. 4 is a side view of the distal tip of the prior art shown in FIG. 3, contacting tissue;

FIG. 5 is a side view of the distal tip of FIG. 2, contacting tissue;

FIG. 6 is a schematic illustration of the gastric positioning device of FIG. 1, being navigated through an enteral pathway of a subject;

FIG. 7 an enlarged view of a distal tip of a gastric positioning device having a curved configuration;

FIG. 8 is a schematic illustration of the distal tip of FIG. 7, being navigated into the stomach of a subject;

FIG. 9A is a block diagram of a force monitoring system of a gastric positioning device;

FIG. 9B is a flowchart depicting use of the force monitoring system of FIG. 9A;

FIG. 10 is a side view of a gastric positioning device including a force monitoring system disposed within a handle, and showing a distal tip of the gastric positioning device engaged with a pylorus of a subject and a sail deployed within a stomach; and

FIG. 11 is a perspective view of a gastric positioning device including a force monitoring system having a force sensor disposed within a distal tip of the gastric positioning device.

Corresponding reference characters indicate corresponding parts throughout the drawings.

### DETAILED DESCRIPTION

The disclosure is directed to a gastric positioning surgical device having an asymmetrical distal tip geometry and/or a force monitoring system for reducing the likelihood of tissue injury (e.g., perforation) during medical procedures (e.g., gastric and bariatric procedures).

Embodiments can include one or more of the following advantages.

In some embodiments, the distal tip of the gastric positioning surgical device has an asymmetrical geometry including a beveled edge. In this configuration, the asymmetrical design and beveled edge increases a surface contact area of the distal tip against tissue for easier buckling under increased insertion force. Thus, as compared to other distal tip configurations, the distal tip of the disclosure is less likely to damage tissue. This can reduce the likelihood of tissue perforation that would interrupt a medical treatment of a subject. The design also helps maintain the length and diameter of the distal tip to minimize stapling over the elongate shaft as compared to devices having tapered tip configurations.

In certain embodiments, the distal tip is curved. The curved distal tip can increase the steerability of the gastric positioning surgical device along a path of least resistance and/or minimize the force necessary for buckling during insertion into a subject for reducing the likelihood of tissue puncture.

In some embodiments, the gastric positioning surgical device includes a force monitoring system including a force sensor for measuring insertion force at the distal tip and an indicator for alerting a clinician of excessive insertion forces. This system can reduce the likelihood of tissue injury as the clinician is notified when tissue stress imposed by the distal tip is approaching that of tissue damage.

In this disclosure, the term “proximal” refers to the portion of a structure closer to a clinician, while the term “distal” refers to the portion of the same structure further from the clinician. As used herein, the term “subject” refers to a human patient or other animal. The term “clinician” refers to a doctor, nurse, or other care provider and may include support personnel.

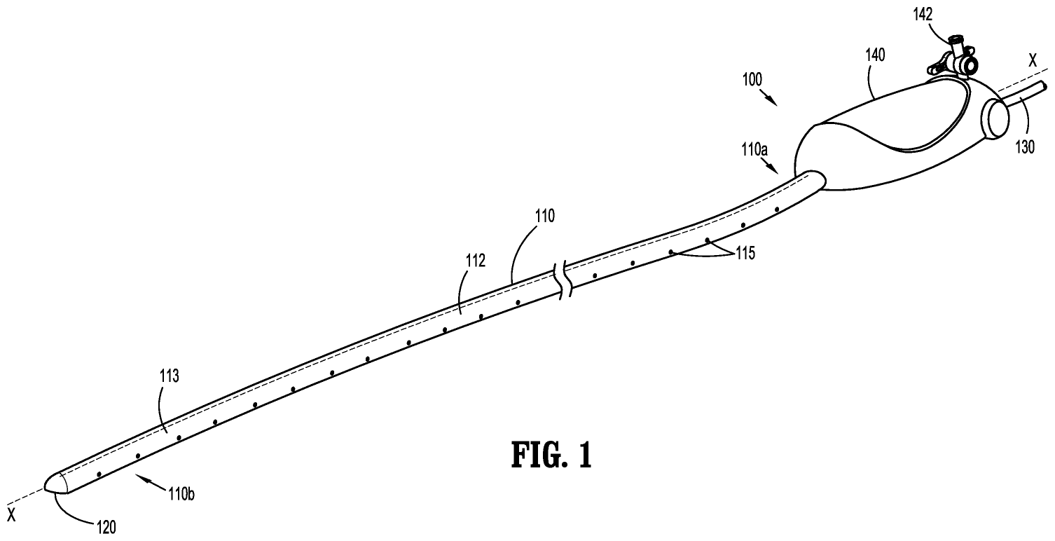

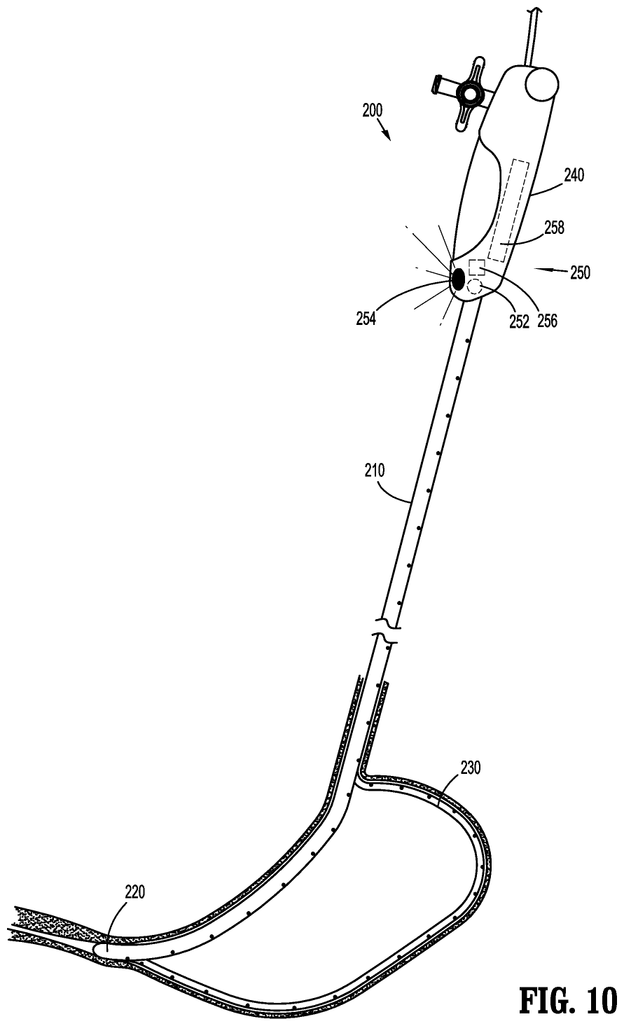

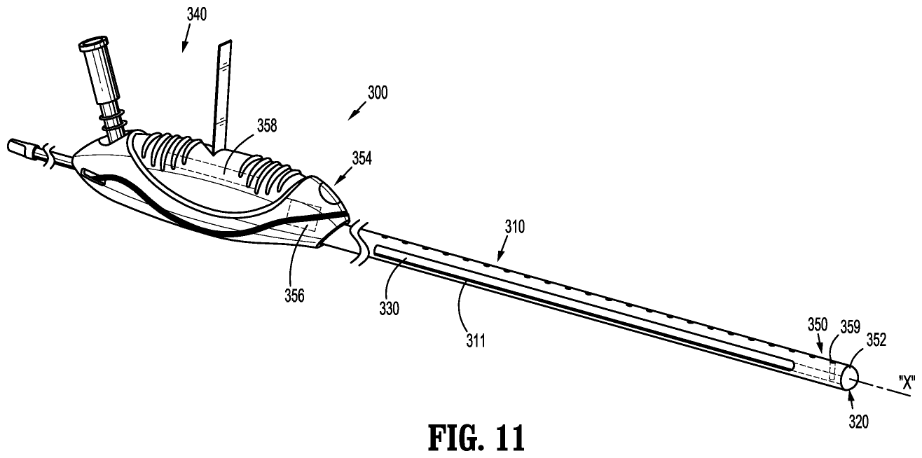

FIG. 1 illustrates a gastric positioning device 100 including an elongate shaft 110, a distal tip 120 distally extending from the elongate shaft 110, a sail 130 supported on the elongate shaft 110, and, optionally, a handle or hub 140 extending proximally from the elongate shaft 110. The sail 130 is movable relative to the elongate shaft 110 between an unexpanded or contracted position, in which the sail 130 is disposed against or within the elongate shaft 110 (e.g., within a window 311 as shown in FIG. 11), and an expanded or deployed position, in which the sail is bowed outwardly from the elongate shaft 110 (see e.g., FIG. 10). Vacuum and/or fluid sources may be coupled to the elongate shaft 110 (e.g., via a luer connector 142 of the handle 140) for the application of, for example, suction, decompression, and/or drainage during medical procedures (e.g., gastric and bariatric procedures).

The elongate shaft 110 can be formed of any material sufficiently flexible to enable the elongate shaft 110 to be advanced trans-orally and maneuvered along a track or enteral pathway of a subject. The elongate shaft 110 can be straight or have a pre-curved configuration (e.g., along its entire length or portion(s) thereof) in the absence of an external stressor.

The elongate shaft 110 has a tubular body 112 defining a central longitudinal axis “X” and includes a proximal end portion 110a and a distal end portion 110b. The tubular body 112 defines a lumen 113 (shown in phantom) extending along and through the length of the tubular body 112, and apertures 115 defined through the tubular body 112 to provide fluid communication between the lumen 113 and an environment exterior to the elongate shaft 110. It is envisioned that the tubular body 112 can have other configurations, such as a non-circular shape (e.g., elliptical or oval) and/or a multi-lumen arrangement.

The proximal end portion 110a of the elongate shaft 110 has an opening (not explicitly shown) fluidly coupled with the luer connector 142 of the handle 140 for operable connection to the vacuum and/or fluid source. Suction may be applied to the elongate shaft 110 through the lumen 113 and the apertures 115 of the tubular body 112 such that the elongate shaft 110 can adhere to tissue (e.g., stomach tissue) due to the apertures 115 directing suction towards the tissue. Fluid (e.g., liquid or gas) may be delivered to, or removed from, the elongated shaft 110 through the apertures 115 and the lumen 113 of the tubular body 112.

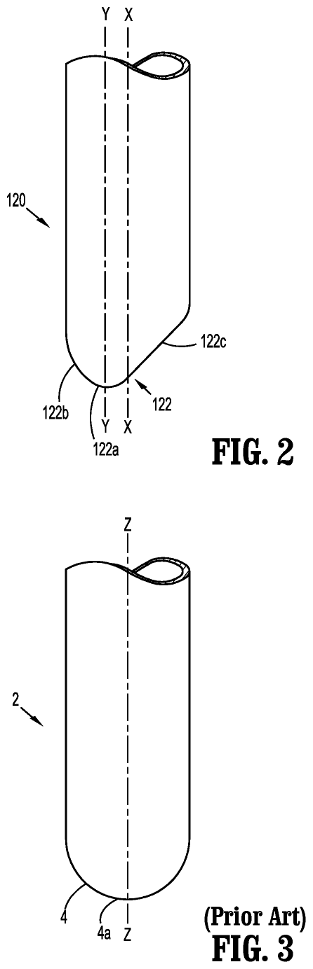

The distal end portion 110b of the elongate shaft 110 includes the distal tip 120. The distal tip 120 is atraumatic and has an asymmetrical geometry for reducing discomfort and/or tissue injury when advancing the elongate shaft 110 through a body cavity or body vessel. As shown in FIG. 2, the distal tip 120 includes a closed distal end 122 having a blunt or rounded apex 122a aligned along a tip axis “Y” that is substantially parallel to, and laterally offset from, the central longitudinal axis “X” of the elongate shaft 110. A curved leading edge or surface 122b and an oblique or beveled edge or surface 122c extend contiguously from the rounded apex 122a such that the distal tip 120 extends asymmetrically about both the tip axis “Y” and the central longitudinal axis “X” of the elongate shaft 110.

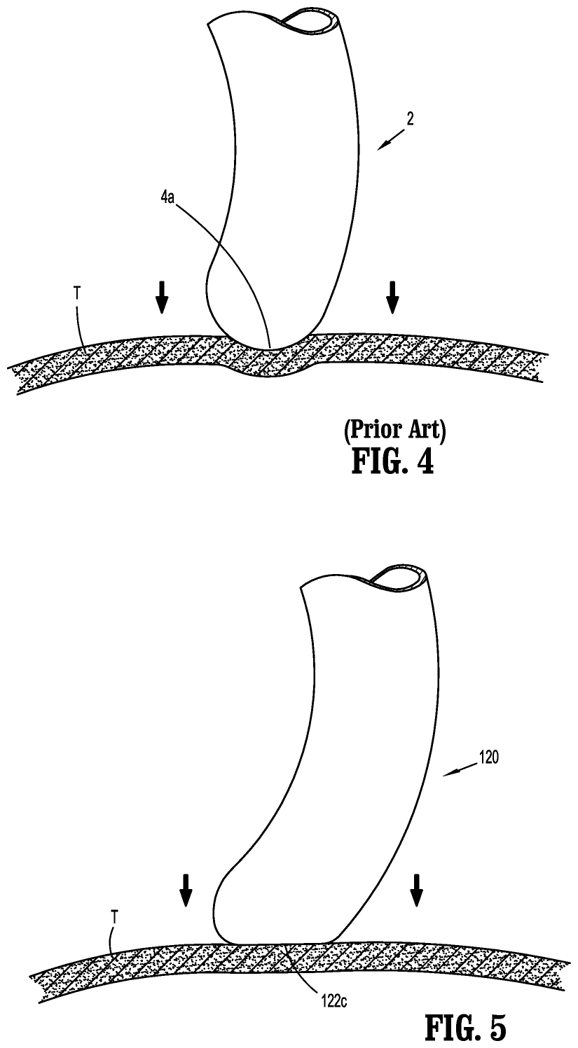

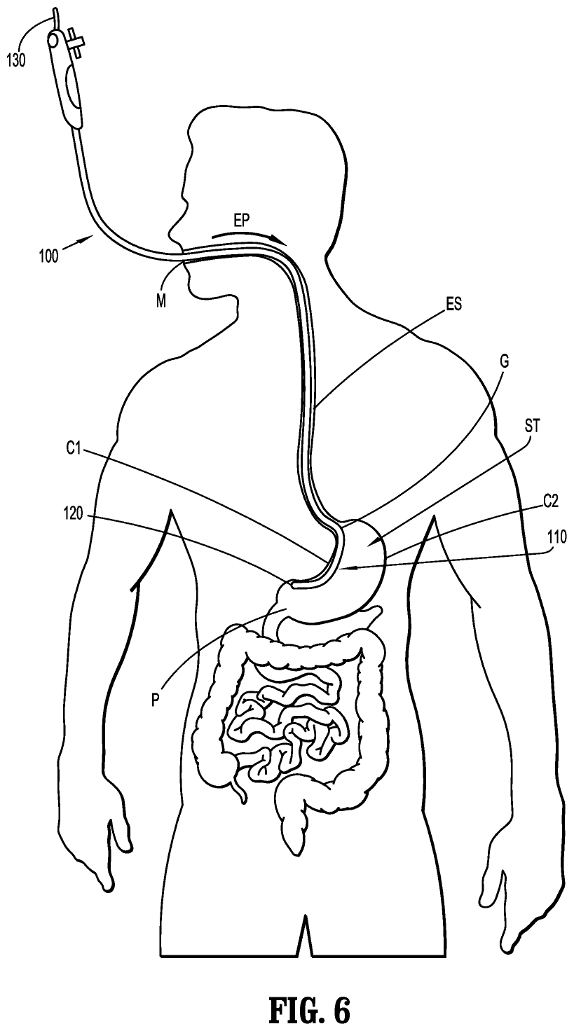

In comparison, as shown in FIG. 3, a prior art distal tip 2 includes a closed distal end 4 having a blunt or rounded apex 4a extending symmetrically about a tip axis “Z” aligned with the central longitudinal axis “X” (FIG. 1) of the elongate shaft 110. In use, as shown in FIG. 4, when the prior art distal tip 2 contacts tissue “T,” the insertion force or direction of force is concentrated in one point at the rounded apex 4a. In contrast, as shown in FIG. 5, when the distal tip 120 of the disclosure contacts tissue “T,” the insertion force or direction of force is spread over a greater tissue contact area across the beveled edge 122c. The increased tissue contact area achieved by the asymmetrical geometry of the distal tip 120 enables consistent directional buckling of the distal tip 120 under resistance along the line of travel of the elongate shaft 110 (e.g., along the central longitudinal axis “X”) resulting in lower insertion forces. Buckling of the distal tip 120 further increases the contact area between the distal tip 120 and the tissue “T” to reduce tissue stress, with lower tissue stress minimizing the change of tissue damage or perforation.

In use, as shown in FIG. 6, the gastric positioning device 100 is inserted into an oral cavity (e.g., a mouth “M”) of a subject and is advanced distally (i.e., caudally) along an enteral pathway “EP” that includes a track that extends between the oral cavity “M” and a stomach “ST” of a subject. When positioned in the stomach “ST” of the subject (e.g., the antrum or lower part of the stomach), the sail 130 can be expanded or bowed outwardly into abutment with the stomach “ST” (e.g., the greater curvature “C2” of the stomach “ST”) and, in turn, pressingly engage the elongate tube 110 with an opposite side of the stomach “ST” (e.g., the lesser curvature “C1” of the stomach “ST”) to aid the clinician in performing a bariatric surgical procedure, such as a sleeve gastrectomy.

During navigation of the elongate shaft 110 through the enteral pathway “EP,” the elongate shaft 110 is guided down the esophagus “ES” and through the gastroesophageal juncture “G” into the stomach “ST.” The gastroesophageal juncture “G” can be challenging to navigate due to, for example, diaphragm interference, changes in the direction of travel of the distal tip 120, physiological differences between the wall tissues of the esophagus “ES” and the stomach “ST,” and/or if the subject has existing medical conditions, such as a hiatal hernia. The distal tip 120 of the gastric positioning device 100, as described above, buckles easier under resistance, such as at the walls of the esophagus “ES” and the gastroesophageal juncture “G” during insertion, thereby minimizing the risk of tissue perforations during advancement into the stomach “ST.”

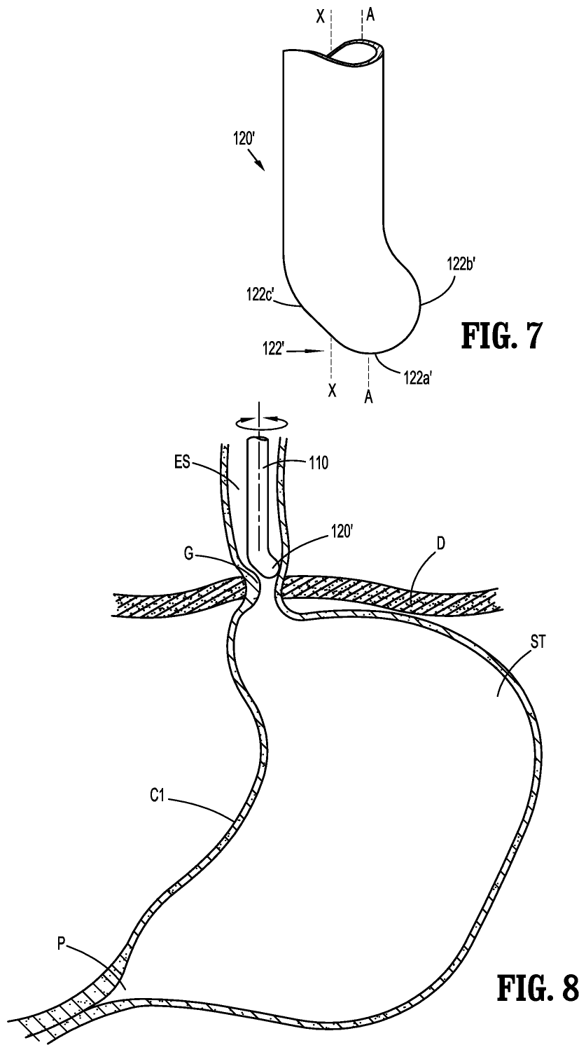

The distal tip of the gastric positioning device may be contoured or curved to enhance steerability (e.g., guiding and/or positioning) of the elongate shaft in challenging conduits. As shown in FIG. 7, a distal tip 120′ is substantially similar to the distal tip 120 of FIG. 2 and includes a closed distal end 122′ having a blunt or rounded apex 122a′ aligned along a tip axis “A” that is substantially parallel to, and laterally offset from, the central longitudinal axis “X” of the elongate shaft 110. A curved leading edge or surface 122b′ and an oblique or beveled edge or surface 122c′ extend contiguously from the rounded apex 122a′ such that the distal tip 120′ extends asymmetrically about both the tip axis “A” and the central longitudinal axis “X” of the elongate shaft 110. The distal tip 120′ is curved such that the distal tip 120′ is angled away from the central longitudinal axis “X” of the elongate shaft 110.

In use, as shown, for example, in FIG. 8, when a clinician reaches an obstruction, such as the tortuous path of the esophagus “ES” or the gastroesophageal juncture “G,” the distal tip 120′ can be rotated (e.g., by rotating the handle 140 (FIG. 1) of the gastric positioning device 100) to reorient the distal tip 120′ during insertion into the stomach “ST” to aid the clinician in finding a path of least resistance and minimizing the insertion force. In another example of use, the distal tip 120′ may engage the pylorus “P” to aid in firmly aligning the elongate shaft 110 with the lesser curvature “C1” of the stomach “ST.”

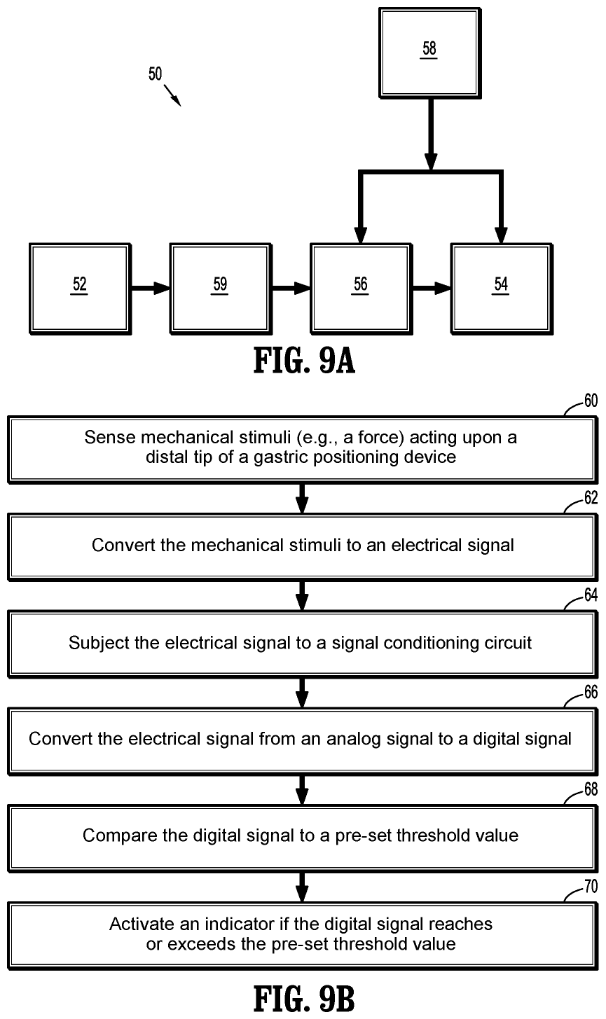

Additionally or alternatively, a force monitoring system may be incorporated into the gastric positioning device for measuring insertion force and providing notice to a clinician when tissue stress imposed by the distal tip of the gastric positioning device is reaching, has reached, and/or exceeds a preset value (e.g., a value associated with tissue strength). As shown in FIG. 9A, a force monitoring system 50 includes a sensor 52, an indicator 54, an integrated circuit 56 (e.g., a microcontroller), a power supply 58, and optionally a receiver 59. The sensor 52 is configured to transform mechanical stimuli into an electrical signal and relay or communicate the electrical signal to the integrated circuit 56 for processing (e.g., converts the electrical signal to a digital signal and/or compares the digital signal to a pre-set or pre-determined digital signal value). In turn, the integrated circuit 56 is configured to communicate with the indicator 54 should the digital signal approach, reach, or exceed the pre-determined digital signal value. The electrical signals from the sensor 52 may first pass through the receiver 59 prior to reaching the integrated circuit 56. The receiver 59 may include a signal conditioning circuit (e.g., for amplifying the electrical signal) and/or an analog-to-digital converter.

As shown in FIG. 9B, in step 60 of using the force monitoring system 50 of FIG. 9A to detect a force at the distal tip 120 (FIG. 1) of the gastric positioning device 100, the force sensor 52 senses mechanical stimuli (e.g., a force acting upon the distal tip 120 of the gastric positioning device 100) and in step 62 the force sensor 52 converts the mechanical stimuli into an electrical signal. Optionally, in step 64 the electrical signal is subjected to a signal conditioning circuit (e.g., the electrical signal is amplified, filtered, etc.) and in step 66, the electrical signal is converted from an analog signal to a digital signal. In step 68, the integrated circuit 56 compares the digital signal (e.g., a signal representing force) to a pre-set or pre-determined threshold value (e.g., a pre-set force threshold value) via a comparison algorithm. In step 70, the integrated circuit 56 communicates with, and activates, the indicator 54 if the digital signal has reached or exceeds the pre-set threshold value.

FIG. 10 illustrates a gastric positioning surgical device 200 including an elongate shaft 210, a distal tip 220 distally extending from the elongate shaft 110, a sail 230 supported on the elongate shaft 210, and a handle 240 proximally extending from the elongate shaft 210. The gastric positioning device 200 is substantially similar to the gastric positioning device 100 of FIG. 1 and will be described with respect to the differences therebetween.

The handle 240 of the gastric positioning device 200 includes a force monitoring system 250 disposed therein. The force monitoring system 250 includes a force sensor 252 (shown in phantom) and an indicator 254. The force sensor 252 detects changes in mechanical stimuli (e.g., pressure or strain) at the distal tip 220, for example, to identify an obstruction or the need to change the direction of advancement of the elongate shaft 210 and/or to recognize differences in tissue type (e.g., differences between the esophagus and the stomach). The indicator 254 may be visual (e.g., a light, such as a light emitting diode), haptic (e.g., force feedback), audible (e.g., a sound), etc., to provide notice to the clinician when an amount of force exceeds a pre-set or pre-determined force threshold value based on, for example, the type of tissue in which the distal tip 220 contacts.

The force sensor 252 may be a strain gage for measuring forces on the distal tip 220 (e.g., pressure applied by tissue acting on the distal tip 220 which, in turn, is applied to the elongate shaft 210 and against the force sensor 252). The force sensor 252 may be electrically coupled to a microcontroller 256 (shown in phantom) disposed within the housing 240 or may be wirelessly coupled to an external processor configured to receive and process electrical signals (e.g., changes in resistance) from the force sensor 252. The electrical signals may include, for example, stress measurements along the distal tip 220 which, in turn, may be converted, via an algorithm, into corresponding tissue stress measurements of the tissue abutting the distal tip 220. The algorithm gates the input based on a pre-set or pre-determined allowable force threshold for a given distal tip diameter to minimize the likelihood of tissue injury.

The indicator 254, shown in the form of an LED, which is powered by a power supply 258 (shown in phantom) disposed in the handle 240, lights up to alert a clinician when an amount of force exceeds the pre-set allowable force threshold. It is envisioned that multiple indicators (e.g., multiple LEDs) may be utilized in the force monitoring system 250 of the disclosure, such as, for example, a first indicator (e.g., a green LED) indicating a safe use condition, a second indicator (e.g., a yellow LED) indicating the force threshold value is being approached, and a third indicator (e.g., a red LED) indicating the force threshold value has been reached or exceeded.

FIG. 11 illustrates a gastric positioning surgical device 300 in accordance with another embodiment of the disclosure. The gastric positioning device 300 includes an elongate shaft 310, a distal tip 320 distally extending from the elongate shaft 310, a sail 330 supported on the elongate shaft 310 within a window 311 defined in the elongated shaft 310, and a handle 340 proximally extending from the elongate shaft 310. The gastric positioning device 300 is substantially similar to the gastric positioning device 100 of FIG. 1 and will be described with respect to the differences therebetween.

The gastric positioning device 300 includes a force monitoring system 350. The force monitoring system 350 includes a force sensor 352 disposed at the distal tip 320 of the gastric positioning device 300 and an indicator 354 disposed within the handle 340. The force sensor 352 measures force (e.g., pressure) exerted on tissue by the distal tip 320 and the indicator 354 alerts a clinician that the force is about to exceed or exceeds a pre-set force threshold value.

The force sensor 352 may be a piezoresistive sensor formed from a piezoresistive material, such as rubber including insulating elastomer and conductive nanoparticles homogeneously dispersed therethrough, shaped to form the distal tip 320. The distal tip 320 may be configured to have the asymmetrical geometry described above with regard to distal tip 120, 220 or may have other configurations, such as a hemispherical shape. The indicator 354 may be visual, haptic, audible, etc., as described above, and is powered by a power supply 358 (shown in phantom) disposed within the handle 340.

The force monitoring system 350 may include a receiver 359 (shown in phantom) disposed within the distal tip 320, proximal of the force sensor 352, for receiving and/or processing electrical signals from the force sensor 352 and communicating the signals to a microcontroller 356 (shown in phantom) disposed within the housing 340. The microcontroller runs an algorithm to convert the electrical signals into corresponding force measurements and compares the force against a pre-set allowable force threshold which is less than the force required to damage tissue, activating the indicator 354 if the pre-set force threshold value is approached or exceeded.

A number of embodiments have been described. Nevertheless, it will be understood that various modifications may be made without departing from the spirit and scope of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, other embodiments are within the scope of the following claims.

What is claimed is:

1. A gastric positioning device comprising:
an elongate shaft having a proximal end portion and a distal end portion, and defining a central longitudinal axis; and
a distal tip extending from the distal end portion of the elongate shaft, the distal tip having an asymmetrical geometry including a closed distal end having a blunt apex aligned along a tip axis laterally offset from the central longitudinal axis.

2. The gastric positioning device of claim 1, wherein the tip axis is parallel to the central longitudinal axis.

3. The gastric positioning device of claim 1, wherein the distal tip includes a beveled edge extending contiguously from the blunt apex.

4. The gastric positioning device of claim 3, wherein the distal tip includes a curved edge.

5. The gastric positioning device according to claim 1, wherein distal tip extends asymmetrically about the tip axis.

6. The gastric positioning device according to claim 5, wherein the distal tip extends asymmetrically about the central longitudinal axis of the elongate shaft.

7. The gastric positioning device of claim 1, wherein the distal tip is curved.

8. The gastric positioning device of claim 1, further comprising a sail supported on the elongate shaft, the sail movable relative to the elongate shaft between an unexpanded position in which the sail abuts the elongate shaft and an expanded position in which the sail is bowed outwardly from the elongate shaft.

9. The gastric positioning device of claim 1, further comprising a handle extending proximally from the elongate shaft.

10. A gastric positioning device comprising:
an elongate shaft having a proximal end portion and a distal end portion;
a distal tip extending from the distal end portion of the elongate shaft;
a handle extending from the proximal end portion of the elongate shaft; and
a force monitoring system including a force sensor for detecting force applied to the distal tip and an indicator for signaling when the force exceeds a pre-set force threshold value.

11. The gastric positioning device of claim 10, wherein the force sensor is disposed within the handle.

12. The gastric positioning device of claim 11, wherein the force sensor is a strain gauge.

13. The gastric positioning device of claim 10, wherein the force sensor is disposed at the distal tip.

14. The gastric positioning device of claim 13, wherein the force sensor is a piezoresistive sensor.

15. The gastric positioning device of claim 13, wherein the distal tip includes a receiver disposed therein, the receiver configured to receive electrical signals from the force sensor.

16. The gastric positioning device of claim 10, wherein the handle includes a microcontroller disposed therein, the microcontroller configured to process electrical signals from the force sensor.

17. The gastric positioning device of claim 10, wherein the indicator is a light.

18. The gastric positioning device of claim 10, wherein the handle includes a power source disposed therein, the power source operably coupled to the indicator.

19. A method of detecting force at a distal tip of a gastric positioning device by a force monitoring system of the gastric positioning device, the method comprising:
sensing a force at a distal tip of a gastric positioning device;
comparing the force to a pre-set force threshold value; and
activating an indicator if the force reaches or exceeds the pre-set force threshold value.

20. The method of claim 19, wherein sensing the force includes transforming mechanical stimuli into an electrical signal, and further comprising processing the electrical signal into a digital value comparable against the pre-set force threshold value.
